(19) United States
(12) Patent Application Publication
(10) Pub. No.: US 2009/0171418 A1
(43) Pub. Date: Jul. 2, 2009

(54) Apparatus for Non-Invasive Stimulation of an Animal

(75) Inventors: Noor Indra Bin Sarif, Singapore (SG); Sze Tat Lim, Singapore (SG); Dennis Yu Guang Han, Singapore (SG); Derek Yong Tang Lim, Singapore (SG); Sharon Puay Ngee Goh, Singapore (SG); Henry Kok Wee Lim, Singapore (SG); Teck Leng Goh, Singapore (SG); Peer M. Sathikh, Singapore (SG); Leo Paul Louis, Singapore (SG); David Picard, Singapore (SG)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: MOLEAC PTE LTD, SINGAPORE (SG)

(21) Appl. No.: 12/162,783

(22) PCT Filed: Jan. 31, 2007
(86) PCT No.: PCT/SG2007/000029

§ 371 Date: Dec. 19, 2008

Related U.S. Application Data
(60) Provisional application No. 60/764,220, filed on Feb. 1, 2006.

(51) Int. Cl. A61N 1/08; A61N 1/00

(52) U.S. Cl. 607/59; 607/2

(57) ABSTRACT

An apparatus for performing non-invasive stimulation of an animal's body, the apparatus comprising: a connector body having a conductor and a pair of stimulators electrically coupled to said conductor, the pair of stimulators being disposed on the connector body to allow the cutaneous surface of the animals body to be engaged therebetween. In use, a power source is electrically coupled to said conductor to transmit an electric current to the pair of stimulators to perform non-invasive stimulation on the animals body as current passes therethrough.

### TECHNICAL FIELD

The invention relates to an apparatus, system and method for performing non-invasive stimulation on a cutaneous surface of an animal's body.

### BACKGROUND

It is known to practice non-invasive stimulation, such as massage and acupuncture, on the cutaneous surface of an animal's body.

Acupuncture is based upon metaphysical concepts of “ch'i” (Qi), a supposed body energy that runs through hyptothesized channels called “meridians”. On these “meridians” are 365 designated acupuncture points that can be used for stimulation to balance “yin and yang” by relieving blockages in the flow of “ch'i”.

Traditional methods of treating medical ailments through acupuncture employ the insertion of needles at the known acupuncture points and the stimulation of the nerves by manually vibrating the needles. However, such transcutaneous nerve stimulation methods inherently possess the risks of causing damage to the body due to accidental bending or breakage of a needle tip.

As an alternative to invasive acupuncture, there are known devices that perform stimulation of the acupuncture points in a non-invasive manner such as applying pulsating electric currents. One known device that uses pulsating electric currents comprises a power supply unit and one or more wires connecting the power supply unit to one or more conducting electrodes. The conducting electrodes are connected to an adhesive patch to hold the conducting electrodes on the acupuncture points of the user to deliver electric currents thereto.

One disadvantage of known devices is that one or more wires that connect the electrodes to the power source may entangle with each other.

Another disadvantage of known devices is that the power supply unit tends to be bulky and therefore not portable. This renders the device inconvenient to use when moving.

Another disadvantage of known devices is that they are voltage controlled rather than current controlled which is essential for the pulse current to transverse through the body.

Another disadvantage of known devices is that the stimulating pulse current produces an unpleasant prickly feeling. Another disadvantage of known devices is that the multiple connections are needed to operate the device, which is generally inconvenient. For example, the electrodes must be connected to the adhesive patches and to the power supply unit, which, in addition to being time consuming

Another disadvantage of known devices is that the multiple cables required for connections may restrict movement of the persons body and thus prevent the carrying of other activities when using the device. The restriction of the persons body is also generally inconvenient.

Another disadvantage of known devices is that the large numbers of wires and the separate power supply may not be aesthetically pleasing and may discourage users from using the device.

Another disadvantage of known devices is that when they are engaged onto a persons body, the electrodes may be randomly placed and not aligned with the location of the desired acupuncture points.

There is a need to provide an apparatus for performing non-invasive stimulation of an acupuncture point on a cutaneous surface of an animals body, that overcomes, or at least ameliorates, one or more of the disadvantages described above.

### SUMMARY

According to a first aspect, there is provided an apparatus for performing non-invasive stimulation of an animal's body, the apparatus comprising: a connector body having an electrical conductor and a pair of stimulators electrically coupled to said electrical conductor, the pair of stimulators being disposed on the connector body to allow the cutaneous surface of the animals body to be engaged therebetween, wherein in use, a power source is electrically coupled to said conductor to transmit an electric current to the pair of stimulators to perform non-invasive stimulation on the animals body as current passes therethrough.

The pair of stimulators may engage an acupuncture point, or the vicinity of an acupuncture point, of the animal's body to perform non-invasive acupuncture stimulation thereon. The pair of stimulators may perform non-invasive stimulation on the cutaneous surface of the animal's body.

The electric current may pass through acupuncture point, or the vicinity of an acupuncture point, of the animal's body. Advantageously, said electric current passing through said acupuncture point, or said vicinity of an acupuncture point, of the animal's body is current controlled.

The connector body may be generally elongate in shape. The pair of stimulators may be respectively disposed at opposite ends of the elongate shaped connector body. The ends of the elongate shaped connector body may have a larger width relative to a middle section of said connector body to allow said ends to pivot about said middle section.

The connector body may comprise a bias for biasing the pair of stimulators towards each other for gripping said animal's body during said non-invasive stimulation. Advantageously, by firmly gripping the animal's body, a substantially even flow of current is transmitted to said animal's body. More advantageously, transmission of an even current across the cutaneous surface of the animal's body prevents, or at least inhibits, arcing of the current or concentration of the current to thereby avoid discomfort to the user during use.

In one embodiment, the bias comprises a leaf spring which may be integrally formed with said connector body. In one embodiment, the connector body is plastic made by molding and the leaf spring may comprise holes which are filled by the plastic when in the molten form. Advantageously, the holes within the leaf spring filled in by the plastic keep the leaf spring in a position relative to the connector body, thereby allowing gripping to the animal's body.

The apparatus may comprise a controller capable of controlling the electrical current. The apparatus may comprise a switch coupled to the controller to turn the electrical current on and off in use. The apparatus may comprise a current modulator coupled to the controller to modulate the intensity of the electric current transmitted in use. The apparatus may comprise a frequency modulator coupled to the controller to modulate the frequency at which the electric current is transmitted in use. The frequency of the electric current may be transmitted from the stimulators to the cutaneous surface of the animals body in the range 2 Hz to 100 Hz.

The connector body may comprise a recess for allowing a battery power source to electrically couple with said conductor. Optionally, a battery power source is integral with said connector body for coupling to said connector.

The connector body may be in any shape such as an enclosed ring or a U-shaped member.

Optionally, one or more indicators are electrically coupled to the controller to indicate information selected from the group consisting of (i) that current is flowing to said stimulators, (ii) that current is not flowing to said stimulators, (iii) the intensity of the current transmitted, (iv) the frequency at which the electric current is transmitted (v) a remaining time for the non-invasive stimulation session and combinations thereof.

The connector body may be made of any suitable material that allows at least part of the connector body to be flexible and resilient. An exemplary material is thermoplastic material such as thermoplastic rubber, flexible foam or combinations thereof.

The apparatus may further comprise an optical coupler electrically coupled to said controller for transmitting and/or receiving, to another optical coupler of another apparatus, information relating to the electric current.

According to a second aspect, there is provided a non-invasive stimulation system comprising: at least two non-invasive stimulation apparatus capable of transmitting an electric current into an animals body, each of said apparatus comprising a controller for generating electric current and an optical coupler capable of transmitting and/or receiving information relating to the electric current; wherein in use, the electric current of the at least two apparatus is synchronized as information relating to the electric current is transmitted between said optical couplers.

Advantageously, it has been found that synchronizing the electric current, and in particular the phase of the electric current, during a non-invasive stimulation session, results in more efficacy than if the electric current is not synchronized.

The electric current may be applied on the cutaneous surface of the animal's body.

In one embodiment, the frequencies of the electric currents of said at least two apparatus are synchronized.

In another embodiment, the phase of the electric currents of said at least two apparatus are synchronized.

In another embodiment, the intensity of the electric currents of said at least two apparatus are synchronized.

According to a third aspect, there is provided a non-invasive stimulation system comprising: at least two apparatus as defined in the first aspect above, each of said apparatus comprising a controller for generating the electric current and a data coupler capable of transmitting or receiving information relating to the electric current; wherein in use, information relating to the electric current is transmitted between said data couplers to indicate whether the electric current of the two apparatus are synchronized or non-synchronized.

The data couplers may be one or more of optical data couplers and wireless data couplers. Exemplary optical data couplers include infrared detectors and infrared transmitters which respectively detect and transmit data in the form of infrared light. Exemplary wireless data couplers include Wi-Fi enabled couplers, Bluetooth enabled couplers, and radio couplers.

According to a fourth aspect, there is provided a method for performing non-invasive stimulation of an animals body, the method comprising the step of: providing a first apparatus according to the first aspect, positioning the pair of stimulators of the first apparatus into engagement on the animals body; and transmitting an electric current to the pair of stimulators.

The method may further comprising the steps of:

providing a second apparatus according the first aspect; and

positioning the second apparatus on the animal's body to concurrently stimulate said animal using both the first and second apparatus.

The method may comprise the step of synchronizing at least one of the frequency, phase and intensity of the electric currents of said first and second apparatus.

The synchronization of the electric currents of said at least two apparatus may occur at the initial stage of stimulation. Advantageously, by synchronizing at the initial stage of stimulation, power is saved and the device is permitted to function autonomously. Furthermore, by synchronizing at the initial stage of stimulation, synchronization errors are reduced or eliminated during the stimulation.

According to a fifth aspect, there is provided a method for performing non-invasive stimulation of an animals body using the apparatus as defined in the first aspect above, the method used to treat conditions selected from a group consisting of smoking addiction, stroke rehabilitation, headache, menstrual cramps, tennis elbow, fibromyalgia, myofascial pain, osteoarthritis, low-back pain, carpal tunnel syndrome, asthma, opiod, food craving, stress, body pain, alcohol addiction and combinations thereof.

According to a sixth aspect, there is provided an apparatus for performing non-invasive stimulation of an animal's body, the apparatus comprising: a connector body having a conductor and at least two stimulators electrically coupled to said conductor, at least part of the connector body being sufficiently flexible to enable engagement by said at least two stimulators on the animal's body and being sufficiently resilient to hold the stimulators in place during use; wherein in use, a power source is electrically coupled to said conductor to transmit an electric current to the at least two stimulators and thereby perform non-invasive stimulation on the animal.

The at least two stimulators may perform non-invasive stimulation on the cutaneous surface of the animals body.

The non invasive stimulation may be performed by said electric current passing between said at least two stimulators in engagement with the animal's body.

The two stimulators may be respectively disposed at opposite ends of the elongate shaped connector body.

In one embodiment, the pair of stimulators are respectively electrodes which are composition of a conductive layer such as carbon mesh with self adhering hydrogel in order to give low resistance and even current distribution.

In one embodiment, the pair of stimulators are respectively metal pads covered with a releasably adhesive material. In another embodiment, the stimulators are respectively carbon mesh covered with self-adhering hydrogel such as, for example, an ECG electrode pad. It should be noted that the dimensions of the stimulator pads may vary depending on the application. For example, if the current is to be localized at a particular point on the animal's body, the stimulator pads will be dimensioned to cover the desired stimulation point. For example, in one embodiment, the stimulator pad is circular in shape having a dimension of about 30 mm, however it is possible to reduce this diameter to concentrate the current by reducing the area (diameter) of the pad. Conversely, if the intensity of the current is to be reduced, the pad area (diameter) can be increased.

### Definitions

The term “biological system” as used herein is intended to refer to a signaling or regulatory network, either chemical or electrical, that may be stimulated by the non-invasive stimulation of an acupuncture point on a cutaneous surface of an animals body, that overcomes, or at least ameliorates, one or more of the disadvantages described above.

The term “controller unit” as used herein is intended to refer to group of electronic systems comprising but not limited to control circuits which may be digital or analog, and intelligent (by incorporating micro controller or similar electronic parts) or simple (by incorporating other means, for example a power source).

As used herein, the term “cun” is intended to mean a unit of distance based on the width of a person's finger so that 1 cun is approximately equal to the distance across the dorsal surface of the middle finger at the proximal interphalangeal joint.

The term “animal” includes humans and non-human animals.

The term “non-invasive” means that the stimulation occurs without any puncture being made in the animal's cutaneous surface while an electric current flows therethrough.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE DRAWINGS

This present invention is now be described by way of non-limiting examples, with reference to the accompanying drawings, in which:

FIG. 1 shows a perspective first side view of an apparatus for performing non-invasive stimulation in accordance with a described embodiment, in which the apparatus is positioned for engagement with the arm of a human;

FIG. 2 shows a perspective second side view of the apparatus of FIG. 1;

FIG. 3 shows a perspective second side view of the apparatus of FIG. 1, in which the apparatus is placed on a flat surface;

FIG. 4 illustrates an electrical schematic diagram of the main components used in the apparatus of FIG. 1.

FIG. 5 illustrates the different electrical current waveforms that can be produced by the apparatus of FIG. 1.

FIG. 6 illustrates the location of four acupuncture points on two human hands and arms.

FIG. 7 shows a front perspective view of a docking station used to charge the batteries of a pair of apparatus of FIG. 1;

FIG. 8 shows a rear perspective view of a docking station of FIG. 7;

FIG. 9 shows a front perspective view of cover for the docking station of FIG. 7;

FIG. 10 shows a pair of the apparatus of FIG. 1 mounted to the docking station of FIG. 7;

FIG. 11 shows a perspective first side view of another apparatus for performing non-invasive stimulation in accordance with a second described embodiment, in which the apparatus is positioned for engagement with the arm of a human;

FIG. 12 shows a perspective second side view of the apparatus of FIG. 11;

FIG. 13 shows a perspective second side view of the apparatus of FIG. 11, in which the apparatus is placed on a flat surface;

FIG. 14 shows a front perspective view of a docking station used to charge the batteries of a pair of apparatus of FIG. 11;

FIG. 15 shows a rear perspective view of a docking station of FIG. 14;

FIG. 16 shows a front perspective view of cover for the docking station of FIG. 14;

FIG. 17 shows a pair of the apparatus of FIG. 11 mounted to the docking station of FIG. 14;

FIG. 18 shows a perspective front view of another apparatus for performing non-invasive stimulation in accordance with a third described embodiment, in which the apparatus is positioned for engagement with the arm of a human;

FIG. 19 shows a perspective rear view of the apparatus of FIG. 18;

FIG. 20 shows a perspective top view of the apparatus of FIG. 18;

FIG. 21 shows a another perspective side view of the apparatus of FIG. 18;

FIG. 22 shows a side view of the leaf spring used in the apparatus of FIG. 18;

FIG. 23 shows a front side view of the leaf spring of FIG. 22;

FIG. 24 shows a rear side view of the leaf spring of FIG. 22;

FIG. 25 shows a perspective side view of a docking station used to charge the batteries of a pair of the apparatus of FIG. 18;

FIG. 26 shows a front perspective side view of a pair of the apparatus of FIG. 18 mounted to the docking station of FIG. 25;

FIG. 27 shows a rear perspective side view of a pair of the apparatus of FIG. 18 mounted to the docking station of FIG. 25.

FIG. 28 shows a schematic diagram of one embodiment of an electrical circuit for delivery of constant current pulses at a specified rate, sequence and duration.

### DESCRIPTION OF DETAILED EMBODIMENTS

Non-limiting examples of a non-invasive stimulation apparatus will be further described in greater detail by reference to specific disclosed embodiments, which should not be construed as in any way limiting the scope of the invention.

Electric Current to Simulate a Selected Acupuncture Point, or the Vicinity of, the Selected Acupuncture Point

The efficacy of using an electric current to perform acupuncture, the technique being known as electro-acupuncture, is thought to derive from the stimulation of the selected acupuncture point on, or in the vicinity of, the selected acupuncture point. Possible physical structures include one or more of a nerve, subcutaneous tissue, muscle fibre and a biological system.

Stimulating the acupuncture point itself or a physical structure on, or in the vicinity of, the selected acupuncture point has been shown to trigger the synthesis and the release of human natural molecules. These natural molecules can act directly on a number of body's self regulation systems, or trigger through biological pathways the modulation of other molecules that will act remotely from the stimulation point on the body homeostasis. including stimulating the body's natural healing abilities and promoting physical and emotional well-being.

As an illustration, acupuncture performed remotely has been documented to affect the parts of the central nervous system related to sensation and involuntary body functions, such as immune reactions and processes whereby a person's blood pressure, blood flow, and body temperature are regulated.

The electrical current is applied at or within about 1, 2, 3, 4 or 5 cm, preferably about 1 to 2 cm and more preferably within about 0.5, 0.6, 0.7, 0.8, or 0.9 cm of the acupuncture point.

In another embodiment where multiple electrodes are used to transmit several electric currents, once each electrode is placed in the desired position in the vicinity of the selected acupuncture point, each electrode is activated simultaneously to provide simultaneous stimulation of the acupuncture points.

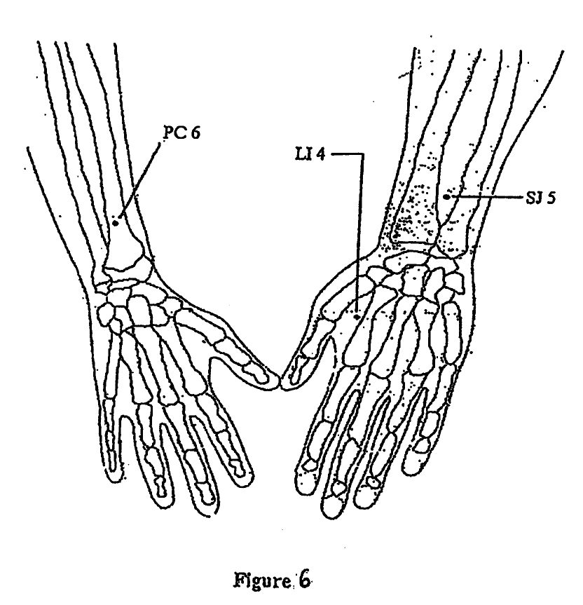

FIG. 6 illustrates the location of four acupuncture points on two human hands and arms: an upper and a lower Hegu (LI 4) acupuncture points respectively located on the back and the palm of one human hand, Wai guan (SJ 5) acupuncture point, and Nei guan (PC 6) acupuncture point. With respect to these four acupuncture points, acupuncture can be performed on, or in the vicinity of, the acupuncture points as follows:

the upper Hegu acupuncture point (LI 4) which is located on the back of each hand where the bones of the thumb and index finger meet;

the lower Hegu acupuncture point (LI 4) on the palm of the hand opposite the upper Hegu acupuncture point (LI 4);

the Wai guan acupuncture point (SJ 5) which is located 2 cun above the transverse crease of dorsum of wrist between the radius and the ulna; and

the Nei guan acupuncture point (PC 6) which is point located 2 cun above the transverse crease of the wrist, between the tendons of m. palmaris longus and m. flexor radialis.

When a first skin-contact electrode transmitting an electric current is in contact on a skin surface on, or in the vicinity of, a selected acupuncture point on the limb of an animal, a second skin-contact electrode should be in contact on the opposite skin surface of the same limb. The positioning of the two skin-contact electrodes in this manner ensures that the electrical current passes through the selected acupuncture point. Preferably the electrical current is applied in the vicinity of a selected acupuncture point to stimulate one or more of the selected acupuncture point, a nerve, subcutaneous tissue, muscle, fibres and a biological system in the vicinity of, the selected acupuncture point.

A range of stimulation strengths can be used to obtain the desired result of curbing the smoker's urge to smoke. The lower limit of this range would be about 1 mA, or at least the subject's threshold. The upper limit of this range would be three times the subject's threshold or 24 mA. A current intensity of 24 mA is typically the upper limit as stimulation above this may cause unpleasant muscle twitching or sensation. In the preferred embodiment of the present invention the transmitted current intensity is approximately two to three times the subject's threshold. It should be appreciated that due to the variability of human sensitivity to electrical current, the actual strength of stimulation used (i.e. the current in mA) is dependent on each subject's pain threshold. While a preferred current intensity range is about 4 to 16 mA, the preferred range of current intensity may need to be adjusted for each individual. For example, sensitivity to pain decreases with age so that a preferred range for an older subject may need to be adjusted to a higher range (e.g. 3 to 7, 4 to 8, 5 to 9, 6 to 10, 5 to 12 mA etc). A range for a younger subject may be 1 to 3, 2 to 4 or 3 to 5 mA, for example.

Due to this variability in pain threshold it will usually be necessary to test a patient's threshold to the electrical stimulation. This is typically done by stimulating the subject with very low current strength (less than 1 mA) and then increasing the strength until the subject first notices the electric stimulation. The point at which the subject first notices the electrical stimulation is the subject's threshold.

A range of electric current frequencies can be used to obtain the desired result of curbing the smoker's urge to smoke. The lower limit of this range would be about 1 Hz with an upper limit of about 120 Hz and between a preferred range of about 2 to about 100 Hz.

Each electro-acupuncture session may consist of a sequence of stimulation under a specific electric current frequency, each specific electric current frequency being about 0.1 to about 30 seconds, and each sequence of such electric current frequency being repeated throughout the session, wherein each session should be repeated thrice a day. It should be noted that the electric current frequency may alternate during said stimulation depending on the conditions to be treated. Exemplary frequencies used for treating such conditions as cigarette smoke addiction are disclosed in international patent publication number WO 2006/043905.

Stimulation of different acupuncture points may be conducted at different electric current intensities and frequencies. Where multiple frequencies are employed the period after which the frequency alternates may differ from point to point.

During the stimulation period, the subject may become adapted to the stimulus (this may typically happen after the first minute or two), with a gradual decline in response. The electrical output may then be adjusted in frequency and/or intensity to resume the sensation.

The strength and duration of stimulation to achieve maximum efficacy should be chosen to maintain patient comfort. The duration of standard electro-acupuncture is dependent on the subject's tolerance of the conditions used and their efficacy. Due to the variable response to the selected conditions between subjects, a successful session may be of any duration from a single application or as long as tolerable. Preferably each session usually lasts at least 1 minute, preferably 10 to 60 minutes, more preferably 10, 35, 20, 25, 30, 35 or 40 minutes and most preferably 20, 25 or 30 minutes.

It has been found that various medical conditions such as, smoking addiction, stroke rehabilitation, headache, menstrual cramps, tennis elbow, fibromyalgia, myofascial pain, osteoarthritis, low-back pain, carpal tunnel syndrome, asthma, opiod, food allergy, stress, body pain and alcohol addiction can be treated by varying the strength, duration, frequency and location of the stimulation.

### Non-Invasive Stimulation Apparatus

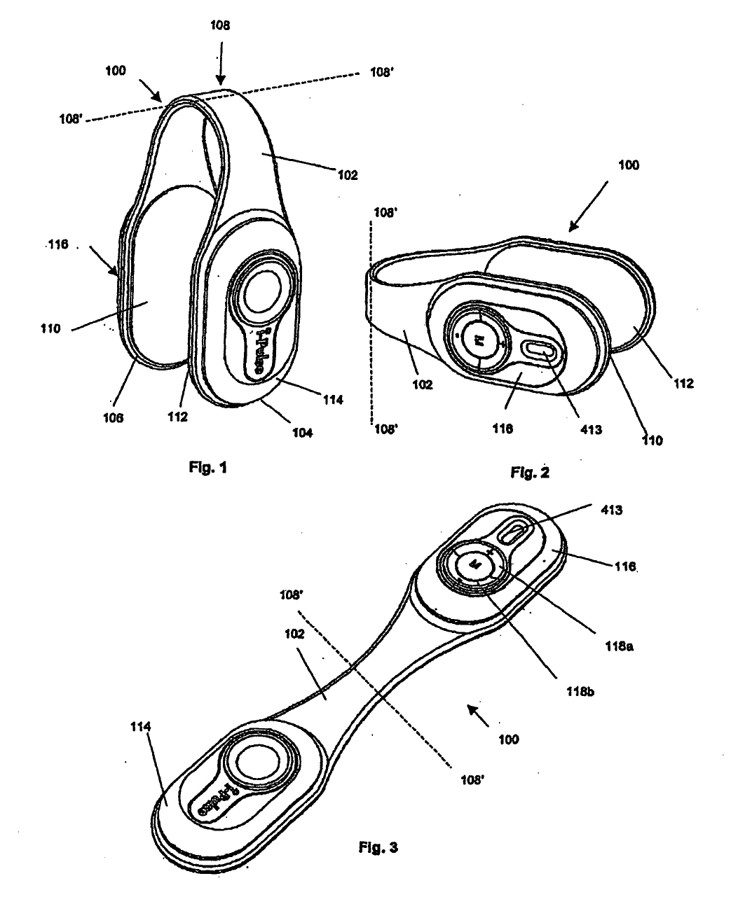

Referring to FIGS. 1, 2 and 3, there is shown an apparatus 100 for performing non-invasive stimulation of an animal's body, such as the Hegu acupuncture point (LI 4) which is shown in FIG. 6. A pair of apparatus 100 can be used to perform non-invasive stimulation of an acupuncture point, or the vicinity of an acupuncture point, on a person's arms. The apparatus 100 can also be used to perform non-invasive stimulation on the cutaneous surface of an animal's body.

The apparatus 100 has a connector body 102 that is generally elongate having two end sections (104,106), and a middle section 108. The width of the middle section 108 is less than the width of the two end sections (104,106) to allow the two end sections (104,106) to pivot about the middle section 108 as shown by dashed line 108′. The connector body 102 is made of a thermo-plastic elastomer, such as SANTOPRENE™ (sold by Advanced Elastomer Systems, Akron, Ohio). The connector body 102 is flexible such that its shape can be deformed to engage with a person's arm or hand when subjected to force by a user but is resilient such that it does not change shape when the force is not applied.

The connector body 102 has a pair of stimulators in the form of skin-contact electrode pads (112, 110). Additionally, a pair of housings (114,116), are respectively provided at the ends (104,106), on respective opposite sides to the skin-contact electrode pads (112, 110). The middle section 108 of the connector body 102 is sufficiently flexible to enable engagement by the skin-contact electrode pads (112,110) on, or in the vicinity of, a persons hand or arm.

To engage a persons hand, the apparatus 100 shown in the non-engagement position shown in FIG. 3, is placed on users persons hand who then bends the respective housings (114,116) toward each other to allow pivotal movement about line 108′. The bending occurs until the apparatus 100 forms a substantially U-shape as shown in FIG. 1 and FIG. 2. The middle section 108 of the connector body 102 is sufficiently resilient to hold the formed U-shape and thereby allow engagement with opposite sides of the person's arm by the respective skin-contact electrode pads (112, 110).

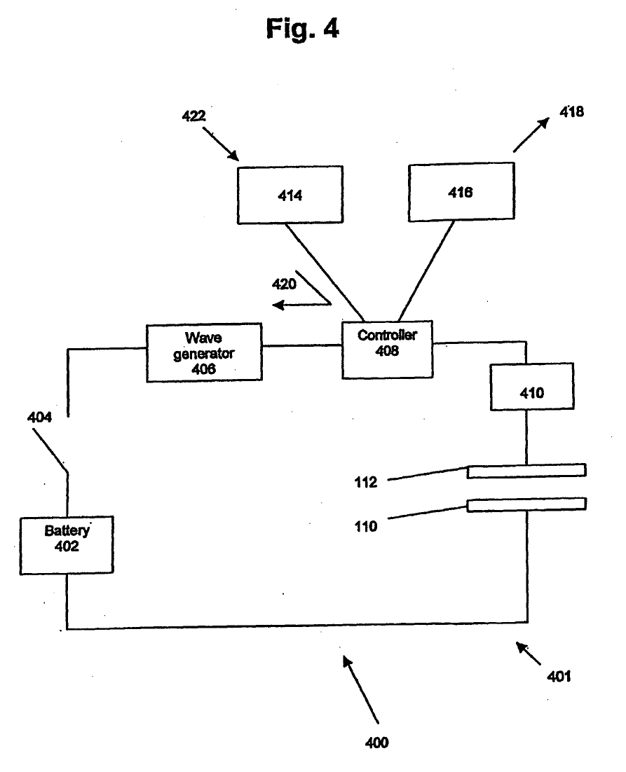

The skin-contact electrode pads (112, 110) are electrically connected to a conductor 401 (shown in FIG. 4) that is resident within the connector body 102. During use, the conductor 401 transmits an electric current to the skin-contact electrode pads (112, 110). The transmitted electric current is then emitted from one of the skin-contact electrode pads (112, 110) onto the skin on one side of the person's arm. The current will enter the skin and pass through the various layers of tissue to exit from the skin on the other side of the person's arm which is in contact with the other skin-contact electrode pad (112, 110). The other skin-contact electrode pad (112, 110) then receives the exiting current. Thus, in this manner, non-invasive stimulation of the person's arm is achieved. Furthermore, because the electric current passes through the person's arm and hand, the electric current advantageously stimulates not just the cutaneous surface of the hand/arm, but also the nerves, tissue layers, muscle, bone, etc., that are below the cutaneous surface. The skin-contact electrode pads (112, 110) are powered, through the conductor 401 by battery power source 402 (shown in FIG. 4), which is resident in housings 114.

The housing 116 also has modulation buttons (118a,118b) that are respectively used to increase (118a) or decrease (118b) the intensity of the current being transmitted to the skin-contact electrode pads (112, 110). The modulation buttons (118a,118b) can also be used to increase or decrease the frequency of the current being transmitted to the skin-contact electrode pads (112, 110) when a mode selection switch (not shown) is present to select whether the modulation buttons 118 modulate the frequency or intensity of the current being transmitted.

FIG. 4 illustrates an electrical schematic diagram of an electrical control system 400 of the main components used in the apparatus 100 of FIGS. 1, 2 and 3. The control system 400 resides within housing 114 and includes the battery power source 402, a switch 404, a wave generator 406, a controller 408, a variable resistor 410, an optical receiver 414, an optical transmitter 416 and the skin-contact electrode pads (112, 110) electrically connected by the conductor 401 that is resident within the connector body 102.

The optical receiver 414 and optical transmitter 416 (not shown in FIGS. 1-3) are disposed such that the optical receiver 414 and the optical transmitter 416 can respectively optically receive a signal from the optical transmitter 416 and transmit a signal optically to the optical receiver 414 of another apparatus 100. The optical receiver 414 and the optical transmitter 416 are used to synchronise the electric current phases being transmitted by one or more apparatus 100 used to performing non-invasive stimulation of acupuncture points, which will be explained further below.

A first LED (Light Emitting Diode) indicator 413 residing on housing 116 lights up when the electric current phase of the apparatus 100 has been synchronised with the electric current phase of another corresponding apparatus 100.

The modulation buttons 118, the optical receiver 414 and the optical transmitter 416 are electrically coupled to the controller 408 (shown in FIG. 4) which is resident within the housing 114.

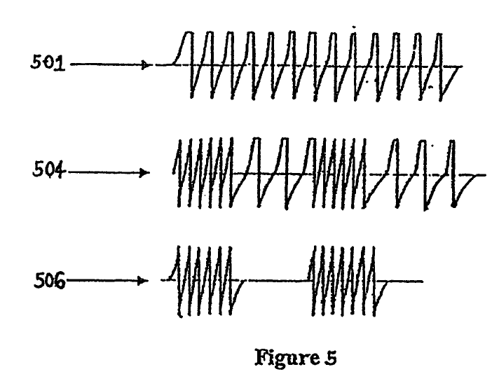

When the apparatus 100 is in use, the power source 402 powers the wave generator 406 to transmit electric current to the skin-contact electrode pads (112, 110). The battery power source 402 also powers the controller 408. The battery power source 402 are rechargeable Li-ion or Li-polymer batteries wherein the apparatus of FIG. 1 is attached to a docking station 700 (shown in FIGS. 5 to 8) to recharge the battery, which will be described further below.

The switch 404 is used to switch the apparatus on or off.

The wave generator 406 is used to output electric currents of different waveforms, such as waveforms 501, 504 and 506 shown in FIG. 5 of selected frequency values. The wave generator 406 may be part of the controller 408 or a separate circuit.

The controller 408 is used to control the operation of the apparatus 100 and is electrically coupled via the conductor 401 to the wave generator 406, the variable resistor 410, the optical receiver 414 and the optical transmitter 416. In one embodiment, the controller 408 used is a microprocessor.

The variable resistor 410 controls the intensity of the electric current being transmitted to the skin contact pads (112,110). When the intensity of the electric current is respectively increased or decreased via the modulation buttons (118a,118b), the controller 408 will send a signal to respectively increase or decrease the resistance of the variable resistor 410.

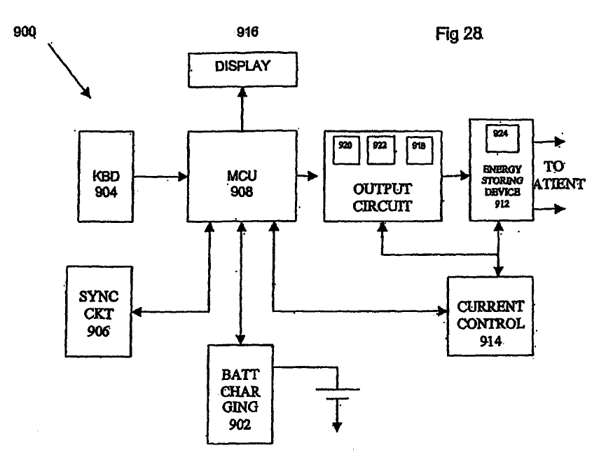

The variable resistor 410 may be of electronic form comprising the OUTPUT CIRCUIT 910 and CURRENT CONTROL 914 circuit as shown in FIG. 28.

The optical receiver 414 is used to receive an optical signal 422 carrying data, such as electric current phase information, optically from an optical transmitter 416 of another corresponding apparatus 100. The optical transmitter 416 is used to transmit an optical signal 418 carrying data, such as electric current phase information, optically to an optical receiver 414 of another apparatus 100.

The skin-contact electrode pads (112, 110) transmit the electric current to perform non-invasive acupuncture on, or in the vicinity of, the desired acupuncture points on the body of an animal.

FIG. 5 illustrates the different electrical current waveforms that can be produced by control system 400; a continuous waveform 501, a sparse and dense wave form 502 or an intermittent wave 503. The overall frequency of stimulus (either by continuous wave or pulses of dense waves) is set similar to the frequency of nerve impulses induced by manual stimulation via twirling or lifting/thrusting of an acupuncture needle.

With reference to again FIGS. 1, 4 and 6, operation of the apparatus 100 to curb a human smoker's urge to smoke will now be described. To curb the smoker's urge to smoke, two apparatus 100 are used in conjunction. For the first apparatus 100, one of the skin-contact electrode pads (112, 110) is to be placed on the upper Hegu acupuncture point (LI 4) of the smoker's left hand while the other skin-contact electrode pad (112, 110) is to be placed on the lower Hegu acupuncture point of the same left hand.

For the second apparatus 100, one of the skin-contact electrode pads (112, 110) is to be placed on the Nei guan acupuncture point (PC 6) of the smoker's right hand while the other skin-contact electrode pad (112, 110) is to be placed on the Wei guan acupuncture point (SJ 5) of the same right hand.

The skin-contact electrode pads (112, 110) of the first apparatus 100 transmit the electric current within the range of about 0.5 to 5 cm of both the upper and the lower Hegu (LI-4) acupuncture points.

The skin-contact electrode pads (112, 110) of the second apparatus 100 transmit the electric current within the range of about 0.5 to 5 cm of the Nei guan acupuncture point (FC 6) and within the range of about 0.5 to 5 cm of the Wai guan acupuncture point (SJ 5).

To increase the efficacy of the stimulation treatment, the frequency and phase of the electric current from the first and the second apparatus 100 have to be the same.

The synchronisation of the phases between the first and the second apparatus 100 electric currents is achieved through the use of the optical receiver 414 and the optical transmitter 416 on each of the first and second apparatus 100.

Before the first and second apparatus 100 are engaged onto the smoker's hands, the first and second apparatus 100 are brought in proximity with each other so that there is optical communication between the first apparatus 100 optical transmitter 416 and the second apparatus 100 optical receiver 414. In the case where the first apparatus 100 acts as the control apparatus, the first apparatus 100 optical transmitter 416 sends an optical signal 218 comprising phase information about the electric current being generated by the first apparatus 100 wave generator 406 to the second apparatus 100 optical receiver 414. The second apparatus 100 optical receiver 414 receives the optical signal 218 and sends a signal 220 to the second apparatus 100 wave generator 406 via the second apparatus 100 controller 408 to synchronise the phase of its generated electric current with the phase of the first apparatus 100 electric current.

The synchronisation of the two electric currents only needs to be done once, thereafter the first and second apparatus 100 are ready to be engaged onto the smoker's hands. It will be appreciated that if the frequency is changed on either the first or second apparatus 100 to treat another condition such as low-back pain, the above synchronisation procedure has to be repeated to synchronise the electric currents of the two apparatus 100. After the two electric currents are synchronised, an LED indicator 413 will light up on both apparatus 100 and both apparatus 100 can then be placed on, or in the vicinity of, the desired acupuncture points.

Referring to FIGS. 7, 8, 9 and 10, there is shown a docking station 700. The docking station 700 has a base portion 702 with a bottom that is sufficiently wide so that the docking station 700 rests stably on the surface the docking station 700 is placed on when the apparatus 100 of FIGS. 1, 2 and 3 is engaged with the docking station 700. The docking station comprises a body 708 with a first side wall 704 and a second side wall 706. The first side wall 704 and the second side wall 706 are disposed at an angle that is offset several degrees from a vertical axis drawn from the bottom of the base portion 702. The tip of the first side wall 704 is disposed a greater distance from the base portion 702 high relative to the tip of the second side wall 706.

The body 708 has recesses 710 that are shaped so as to accommodate the connector body 102 and the end sections (104, 106) of the apparatus 100. The recesses 710 can have male-shaped terminals (not shown) to engage with a corresponding female-shaped terminal (not shown) along the connector body 102 of the apparatus 100. The male shaped terminal and the female-shaped terminals allow the docking station 700 to charge the power source 402 of the apparatus 100 when the apparatus 100 is engaged with the recess 710.

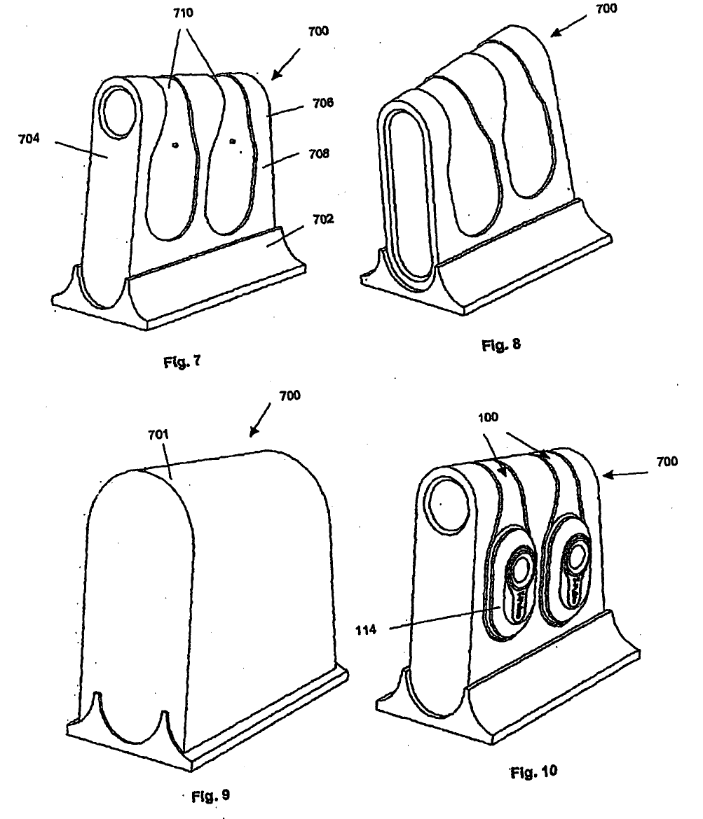

A cover 701 is provided that engages with the base portion 702 to prevent dust from settling onto the body 708 as shown in FIG. 9.

The docking station 700 stores the apparatus 100 when the apparatus 100 is not in use as illustrated in FIG. 10. The docking station 700 can also be connected to a power mains (not shown) so as to facilitate the charging of the power source 402 of the apparatus 100.

It will be appreciated that the apparatus 100 may take other shapes and forms. For example, referring to FIGS. 11-13, there is shown a second apparatus 1100 for performing non-invasive stimulation of an acupuncture point on a humans hand and arm, such as the Hegu acupuncture point (LI 4) which is shown in FIG. 6. The apparatus 1100 functions in exactly the same way and has the same features as the apparatus described with reference to FIGS. 1-3 above, except that the numerals for the same features are numbers by an additional 1000.

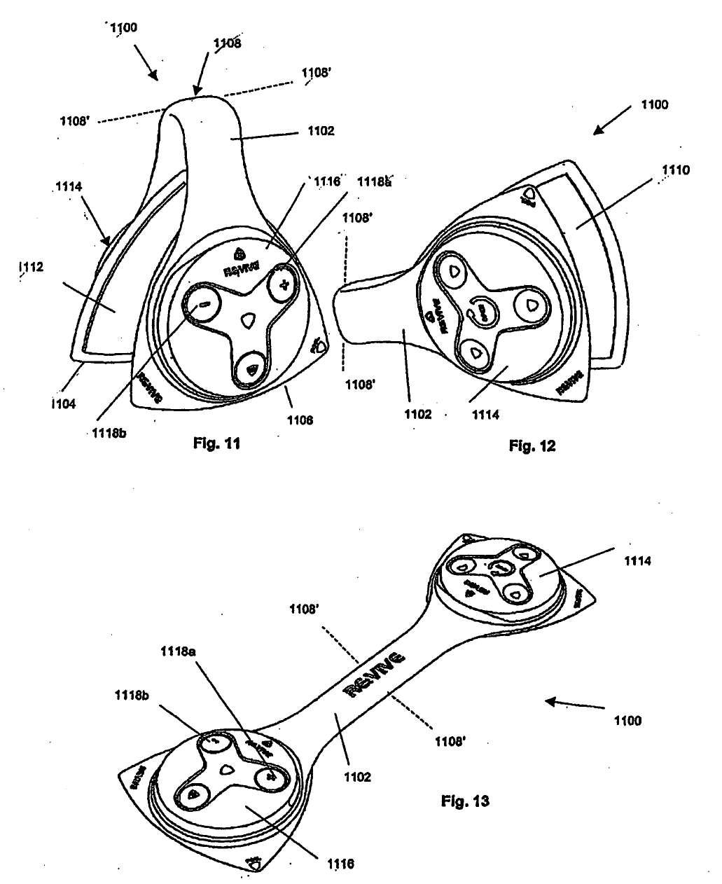

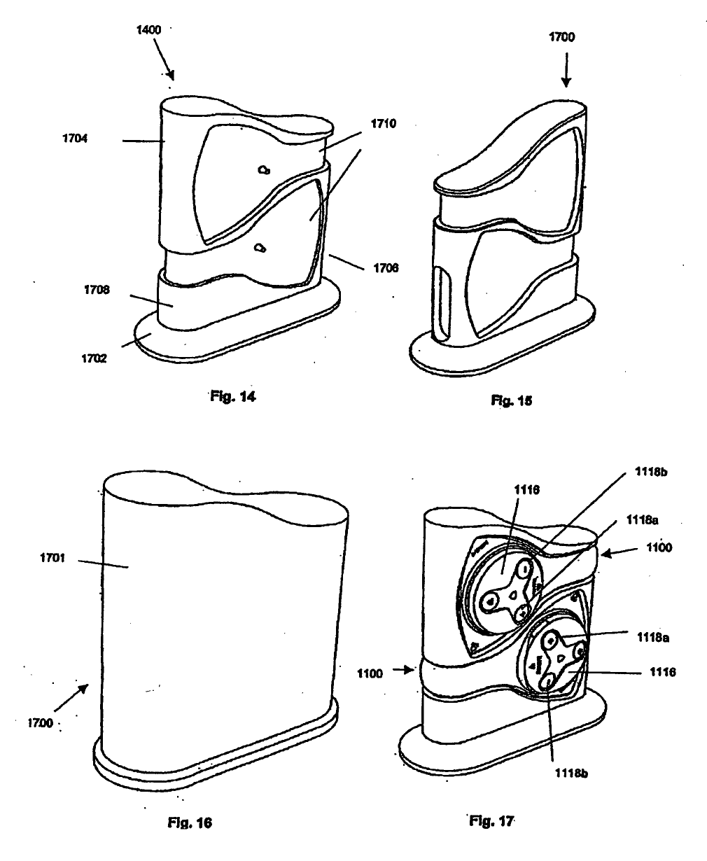

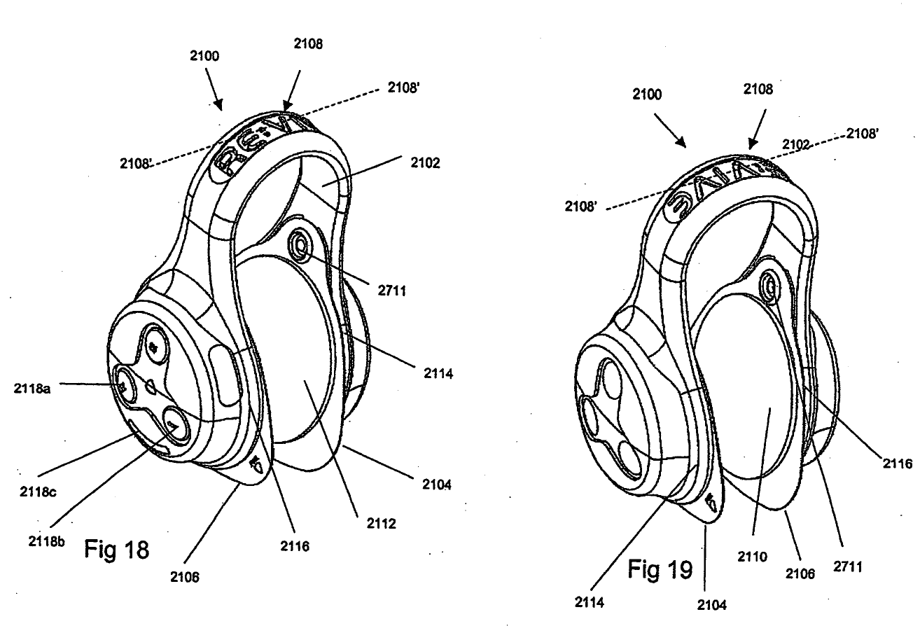

Additionally, the docking station 700 may take other shapes and forms. Referring to FIGS. 14-17, there is shown a second docking station 1700, which functions in exactly the same way and has the same features as the docking station 700 described with reference to FIGS. 1-3 above, except that the numerals for the same features are numbers by an additional 1000.

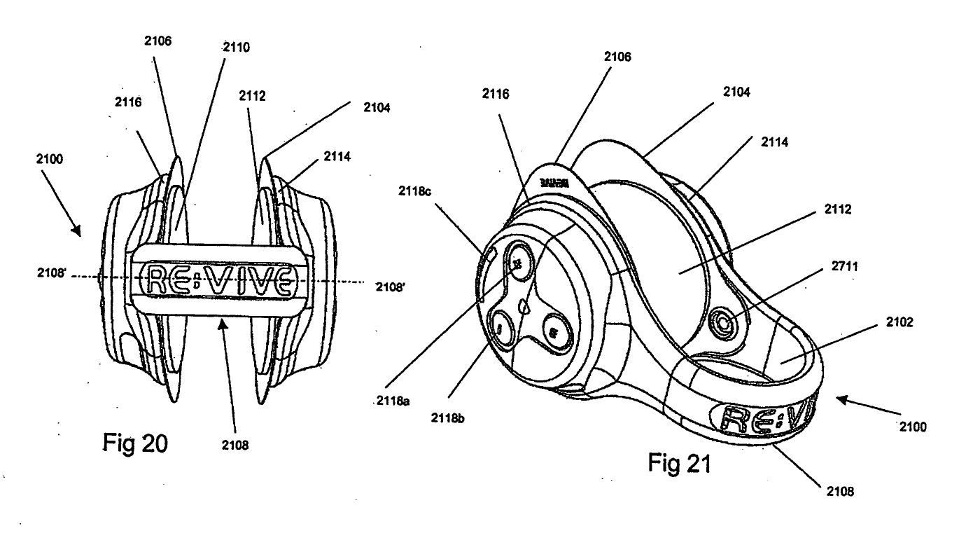

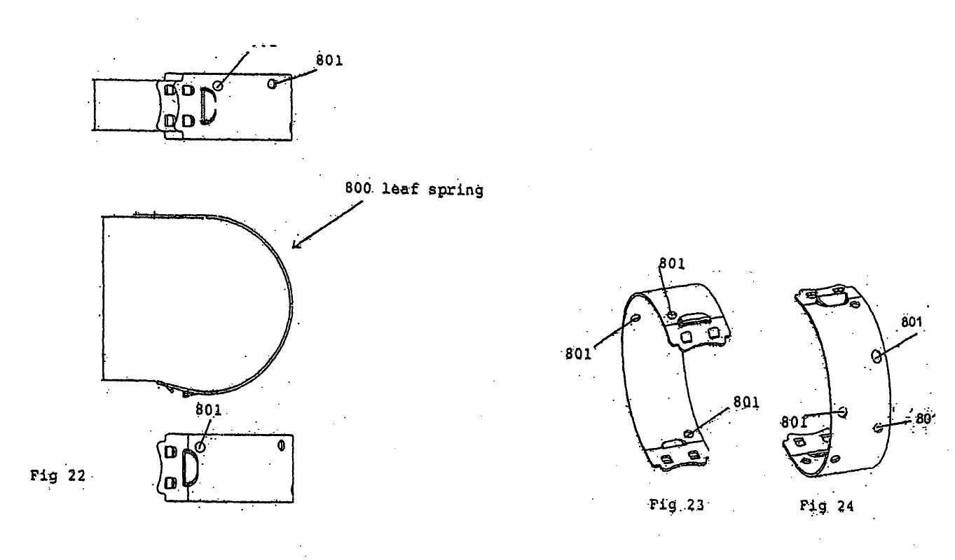

Referring to FIGS. 18-21, there is shown a third apparatus 2100 for performing non-invasive stimulation of an acupuncture point on a human hand and arm, such as the Hegu acupuncture point (LI 4) which is shown in FIG. 6. The apparatus 2100 functions in exactly the same way and has the same features as the apparatus described with reference to FIGS. 11 -13 above, except that the numerals for the same features are numbers by an additional 1000 and that the apparatus 2100 comprises a a leaf spring 800 (FIGS. 22-24) intergreally formed with the connector body 2102. In one embodiment, the connector body 2102 is plastic made by molding and the leaf spring 800 may comprise holes 801 which are filled by the plastic when in the molten form. The leaf spring 800 may be made from any suitable leaf spring metal such as is known in the art. Typical dimensions are about 0.5 mm to about 1 mm thickness, a width of about 0.5 mm to about 15 mm and a length of about 50 to about 150 mm.

In addition to the modulation buttons (2118a, 2118b) that are respectively used to increase (2118a) or decrease (2118b) the intensity of the current being transmitted to the skin-contact electrode pads (2112, 2110), the apparatus includes a meter 2118C for indicating the magnitude of the current in milli amps.

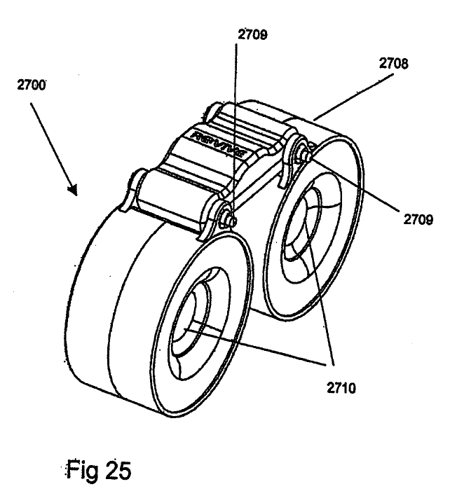

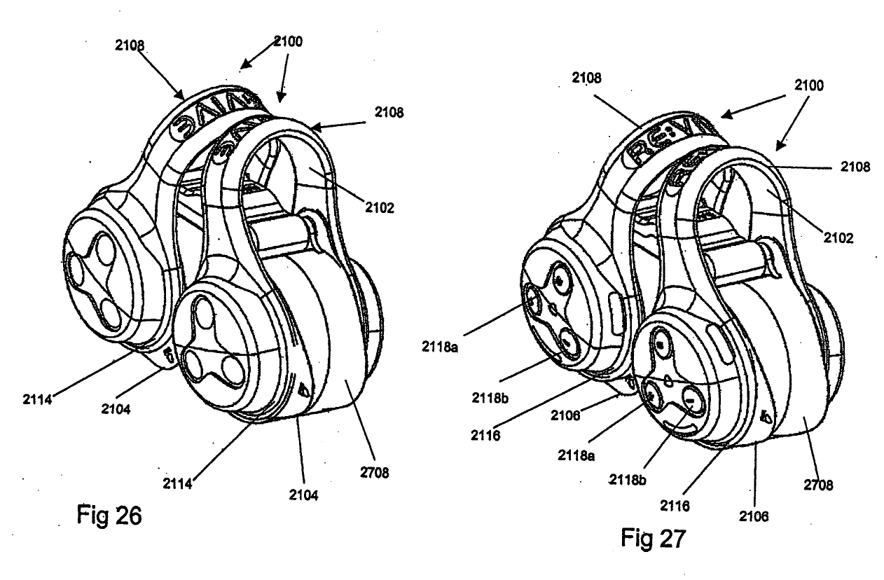

Additionally, there is shown a third docking station 2700 in FIGS. 25-27, which functions in exactly the same way and has the same features as the docking station 1700 described with reference to FIGS. 14-17 above, with the exception that the docking station includes male electrodes 2709 for mating with corresponding female electrodes 2711 located on each of the apparatus (refer to FIG. 18, FIG. 19 and FIG. 21). The male electrodes 2709 not only allow recharging of the battery located within the 2102, but also allow neatly engage the apparatus 2100 during recharging.

FIG. 28 illustrates an electrical schematic diagram of an electrical control system 900 for delivery of constant current pulses to the patient at a specified rate, sequence and duration by non-magnetic means. The MCU (Micro Controller Unit) 908 controls the pulse sequence, rate, duration and the output current through the CURRENT CONTROL 914 circuit. In addition, the MCU 908 handles keyboard (KBD) controller 904 inputs, DISPLAY 916 and SYNChronizing 906 of the pulse delivery with the other unit of the pair of apparatus. The KBD 904 is used as an interface for receiving input from the patient to control the MCU 908. The OUTPUT CIRCUIT 910 converts the low level signals from the MCU 908 to a level appropriate for the particular patient. It also sends the output current feedback to the CURRENT CONTROL 914 circuit. The ENERGY STORING DEVICE 912 bypasses the initial surge current. The unit operates from a Li-ion/Li-polymer battery and it is charged through the BATT CHARGING 902 circuit.

To have current flowing through an animal's body, which has a naturally high contact resistance, a high voltage has to be used. In prior art devices, transformers are employed to step up low voltages to high voltages, but transformers are bulky and therefore impractical for use in portable devices.

The electrical control system 900 achieves the high voltage required to send a constant current by employing a capacitor element 918, a MOSFET switch 920 and an inductor device 922 in the OUTPUT CIRCUIT 910. The capacitor element 918, the MOSFET switch 920 and the inductor device 922 achieve a flyback effect in allowing the circuit 900 to deliver high voltage to the animal body. The MOSFET switch 920 controls the inductor device 922 to switch on the capacitor element 918 to store energy. When the capacitor element 918 has stored sufficient energy to achieve the high voltage required to overcome the contact resistance of the animal's body and thereby send a current pulse through the human skin, the MOSFET switch 920 will activate the inductor device 922 to switch off the capacitor element 918. The MCU 908 will then control when the capacitor element 918 releases the stored energy required to deliver the constant current pulse through the animal body. As the capacitor element 918, the MOSFET switch 920 and the inductor device 922 are compact devices, they are readily employed for use in a portable device.

In one embodiment, the energy storing device 912 uses a wave shaping circuit 924 that supplies an initial surge current to the CURRENT CONTROL 914 circuit, the initial surge current being required by the CURRENT CONTROL 914 circuit to supply a stimulating pulse current to the animal's body. Thus the ENERGY STORING DEVICE 912 prevents the initial surge current to flow through the animal's body thereby avoiding a pricking feeling experienced by an animal when a current is applied.

In one embodiment, the capacitor element 918 has a rating of 2.2 uF, 100V. The capacitor element 918 can store energy to generate a minimum voltage of 25V to a maximum voltage of 50V. It will be appreciated that capacitors with capacitance ratings of about 2.0 to about 2.2 uF and voltage ratings of about 90 to 110V can also be used.

The OUTPUT CIRCUIT 910 includes a wave shaping circuit in order to avoid the unpleasant prickly feeling mentioned earlier. In one embodiment, an ENERGY STORING DEVICE 912 is used to supply the initial surge current required by the CURRENT CONTROL 914 circuit when applying the stimulating pulse current. This arrangement prevents the initial surge current, which causes the unpleasant prickly feeling, from flowing through the patient's body.

The OUTPUT CIRCUIT 910 incorporates a H-bridge switching circuit to provide bi-phasic current.

Advantageously, the electrical control system is in a miniature form without having its efficacy compromised.

It will be appreciated that the disclosed apparatus (100, 1100, 2100) disclosed above, provide a useful alternative to known invasive acupuncture devices. The disclosed apparatus (100, 1100, 2100) provide a single unitary unit that is portable and easy to use. For example the apparatus (100, 1100, 2100) do not require wires outside of the connector body 102 for connecting to the skin-contact electrode pads (112,110). Accordingly the disclosed apparatus (100, 1100, 2100) do not have wires that entangle with each other.

The disclosed apparatus (100, 1100, 2100), and particularly the apparatus 2100 with the leaf spring, ensures firm adhesion of the stimulator pads to the user's arm. This ensures that the stimulation of the user's arm is stimulated over the whole area of the pad. Furthermore, application of an adhesive material such as a self-adhesive hydrogel promotes adhesion of the stimulators to the users arm. The firm adhesion to the user's arm also reduces, or prevents, arcing of the current or concentration of the current on the users arm, thereby avoiding discomfort to the user.

The electrical control system 900 of the apparatus (2100) allows a constant current to be applied to a users body, thereby resulting in enhanced stimulation of the user. The constant current can be delivered without the use of a transformer which results in the control system 900 being minaturised in the disclosed apparatus (2100). The disclosed control system 900 is able, without use of a transformer, to generate a relatively high voltage which overcomes the natural resistance of a user's arm, thereby being converted to a current that passes into the user's body.

The disclosed apparatus (100, 1100, 2100) has a power source that is integral with the connector body 102 and therefore avoids the problem of known devices which utilize a bulky power supply unit. The integral power source enhances the portability of the disclosed apparatus (100, 1100, 2100) and makes it convenient to use.

The flexible and resilient middle section 108 of said connector body being flexible, yet resilient, to allow engagement by the skin-contact electrode pads (112,110) on acupuncture points of a human's arm and hand, thereby nullifying the need to use any form of adhesive patch to ensure adherence to a persons skin. This avoids possible painful removal as in known devices.

As the parts of the disclosed apparatus (100, 1100, 2100) are formed into a single integral body, multiple connections are not needed, thereby enhancing the users inconvenience. In this regard, it is not necessary for the skin-contact electrode pads (112,110) to be connected to adhesive patches and to the power supply unit. The user is not restricted in his or her bodies movement.

The disclosed apparatus (100, 1100, 2100) avoids the disadvantage of known devices in that a large numbers of wires and a separate power supply are not necessary. This makes the apparatus aesthetically pleasing and may encourages users to use the device.

Although the disclosed apparatus (100, 1100, 2100) have been disclosed as being able to engage a persons hand arm, it will be appreciated that other apparatus can be used to engage other parts of the users body assuming that the dimensions of the apparatus are adequate.

Furthermore, although the disclosed apparatus (100, 1100, 2100) has been disclosed as operating at a frequency range that will assist in the inhibition of a smokers urge to smoke, other frequencies can be used to affect other indications. For instance, the disclosed apparatus (100, 1100, 2100) can be used to treat medical indications such as smoking addiction, stroke rehabilitation, headache, menstrual cramps, tennis elbow, fibromyalgia, myofascial pain, osteoarthritis, low-back pain, carpal tunnel syndrome, asthma, opiod, food craving, stress, body pain and alcohol addiction.

Although the disclosed apparatus (100, 1100, 2100) has been described above as applying to non-invasive acupuncture, the disclosed apparatus (100, 1100, 2100) could be used to perform non-invasive stimulation that does not involve acupuncture. For example, the disclosed apparatus may be used to in e-stimulation and transcutaneous electrical nerve stimulation (TENS).

Accordingly, it will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. An apparatus for performing non-invasive stimulation of an animal's body, the apparatus comprising:
a connector body having an electrical conductor and a pair of stimulators electrically coupled to said electrical conductor, the pair of stimulators being disposed on the connector body to allow the cutaneous surface of the animals body to be engaged therebetween,
wherein in use, a power source is electrically coupled to said conductor to transmit an electric current to the pair of stimulators to perform non-invasive stimulation on the animal's body as current passes therethrough.

2. The apparatus as claimed in claim 1, wherein the connector body is generally elongate in shape.

3. The apparatus as claimed in claim 1, wherein the connector body comprises a bias for biasing the pair of stimulators towards each other for gripping said animal's body during said non-invasive stimulation.

4. The apparatus as claimed in claim 3, wherein said bias comprises a leaf spring coupled to said connector body.

5. The apparatus as claimed in claim 4, wherein the pair of stimulators are respectively disposed at opposite ends of the elongate shaped connector body.

6. The apparatus as claimed in claim 1 comprising a controller capable of controlling the electrical current.

7. The apparatus as claimed in claim 6, comprising one or more of the following: (a) a switch coupled to the controller to turn the electric current on and off in use; (b) a current modulator coupled to the controller to modulate the intensity of the electric current transmitted in use; and (c) a frequency modulator coupled to the controller to modulate the frequency at which the electric current is transmitted in use.

8. The apparatus as claimed in claim 1, wherein the connector body is in the form of an enclosed ring or a U-shaped member.

9. The apparatus as claimed in claim 6, wherein one or more indicators are electrically coupled to the controller to indicate information selected from the group consisting of (i) that current is flowing to said stimulators, (ii) that current is not flowing to said stimulators, (iii) the intensity of the current transmitted, (iv) the frequency at which the electric current is transmitted (v) a remaining time for the non-invasive stimulation session and combinations thereof.

10. The apparatus as claimed in claim 6, comprising a data coupler electrically coupled to said controller for transmitting and/or receiving, to another coupler of another apparatus, data information relating to the electric current.

11. The apparatus as claimed in claim 10, wherein said data coupler is one of an optical data coupler or wireless data coupler.

12. A non-invasive stimulation system comprising:
at least two non-invasive stimulation apparatus capable of transmitting an electric current into an animal's body, each of said apparatus comprising a controller for generating electric current and an data coupler capable of transmitting and/or receiving data information relating to the electric current;
wherein in use, information relating to the electric current is transmitted between said data couplers to indicate whether the electric current of said two apparatus is synchronized or non-synchronized.

13. The system as claimed in claim 12, wherein said data couplers are one of optical data couplers and wireless data couplers.

14. The system as claimed in claim 12, wherein at least one of the frequency, phase and intensity of the electric currents of said at least two apparatus are synchronized.

15. The system as claimed in claim 14, said synchronization of the electric currents of said at least two apparatus occurs at the initial stage of stimulation.

16. The system as claimed in claim 12, wherein said apparatus further comprises a connector body having an electrical conductor and a pair of stimulators electrically coupled to said electrical conductor, the pair of stimulators being disposed on the connector body to allow the cutaneous surface of the animals body to be engaged therebetween.

17. The system as claimed in claim 16, wherein the connector body comprises a bias for biasing the pair of stimulators towards each other for gripping said animal's body during said non-invasive stimulation.

18. The system as claimed in claim 17, wherein said bias comprises a leaf spring coupled to said connector body.

19. The system as claimed in claim 18, wherein the pair of stimulators are respectively disposed at opposite ends of the connector body.

20. The system as claimed in claim 16, wherein the connector body is in the form of an enclosed ring or a U-shaped member.

21. A method for performing non-invasive stimulation of an animal's body, the method comprising the step of:
providing a first apparatus according to claim 1;
positioning the pair of stimulators of the first apparatus into engagement on the animal's body; and
transmitting an electric current to the pair of two stimulators.

22. The method as claimed in claim 21, wherein the pair of stimulators engage an acupuncture point, or the vicinity of an acupuncture point, of the animal's body to perform non-invasive stimulation thereon.

23. The method as claimed in claim 21 further comprising the steps of:
providing a second apparatus according to claim 1; and
positioning the second apparatus on the animal's body to concurrently stimulate said animal using both the first and second apparatus.

24. The method as claimed in claim 23, comprising the step of synchronizing at least one of the frequency, phase and intensity of the electric currents of said first and second apparatus.

25. The method as claimed in claim 24, wherein said synchronization of the electric currents of said at least two apparatus occurs at the initial stage of stimulation.

26. A method for performing non-invasive stimulation of an animals body using the apparatus of claim 1, the method being used to treat a condition selected from a group consisting of smoking addiction, stroke rehabilitation, headache, menstrual cramps, tennis elbow, fibromyalgia, myofascial pain, osteoarthritis, low-back pain, carpal tunnel syndrome, asthma, opiod, food craving, stress, body pain, alcohol addiction and combinations thereof.

27. An apparatus for performing non-invasive stimulation of an animal's body, the apparatus comprising:
a connector body having a conductor and at least two stimulators electrically coupled to said conductor, at least part of the connector body being sufficiently flexible to enable engagement by said at least two stimulators on the animal's body and being sufficiently resilient to hold the stimulators in place during use;
wherein in use, a power source is electrically coupled to said conductor to transmit an electric current to the at least two stimulators and thereby perform non-invasive stimulation on the animal.

28. An apparatus for performing non-invasive stimulation of an animal's body, the apparatus comprising:
a connector body having an electrical conductor and a pair of stimulators electrically coupled to said electrical conductor, the pair of stimulators being disposed on the connector body to allow the cutaneous surface of the animals body to be engaged therebetween; and
an electrical circuit coupled to said electrical conductor for supplying a electrical current sufficient to overcome contact resistance of an animal's body,
wherein in use, a power source is electrically coupled to said selected electrical circuit to supply said current to said pair of stimulators to perform said non-invasive stimulation on the animal's body.

29. An apparatus as claimed in claim 28, wherein said electrical circuit is configured to supply a substantially constant electrical current to said animals body.

30. An apparatus as claimed in claim 28 or 29, wherein said electrical circuit comprises a capacitor capable of storing sufficient energy to generate said electrical current to overcome contact resistance of said animal's body.

31. An apparatus as claimed in claim 30, said capacitor stores said electrical energy to generate a voltage of 25 volts to 50 volts.

32. An apparatus as claimed in claims 30 or 31, wherein said capacitor has a capacitance rating of about 2.0 to about 2.2 uF.

33. An apparatus as claimed in claim 32, wherein said capacitor has a voltage rating of about 90 to 110V.

34. An apparatus as claimed in claims 28 to 33, wherein said electric circuit comprises an energy storing device for supplying an initial surge current used to generate said electrical current sufficient to overcome contact resistance of an animal's body.

35. An apparatus as claimed in claim 34, wherein said energy storing device comprises a wave shaping circuit for supplying an initial surge current to a current control circuit used to generate said electrical current sufficient to overcome contact resistance of an animal's body.
